SEQUENCE LISTING (1 sequence)

SEQ ID NO: 1 - protein, 176 aa, T4 bacteriophage
MTIKTKAELFNKWKPLLEGEGLPEIANSKQAIIAKIFENQEKDFQTAPEYKDEKIAQAFGSFLTEAEIGGDHGYNATNIAAGQTSGAVTQIGPAVMGMVRRAIPNLIAFDICGVQPMNSPTGEVFALRAVYGKDPVAAGAKEAFHPMVGPDAMFSGQGAAKKFPALAASTQTTVGD

(19) United States
(12) Patent Application Publication
(10) Pub. No.: US 2007/0037258 A1
(43) Pub. Date: Feb. 15, 2007

(54) Methods and compositions for enhancing protein folding

(75) Inventor: Larry Snyder, East Lansing, MI (US)

Correspondence Address: Medlen & Carroll, LLP;Suite 350, 101 Howard Street, San Francisco, CA, 94105, US

(73) Assignee: The Board of Trustees Operating Michigan State University, East Lansing, MI (US)

(21) Appl. No.: 11/199,782

(22) Filed: Aug. 9, 2005

(51) Int. Cl. C07K 14/47; C07K 14/195; C07H 21/04; C12P 21/04; C12N 1/21; C12N 15/74

(52) U.S. Cl. 435/69.7; 435/488; 435/252.33; 530/350; 536/23.5

(57) ABSTRACT

The present invention relates to methods and compositions for enhancing protein folding. In particular, the present invention relates to methods for directing recombinantly expressed proteins to protein folding chaperones.

### FIELD OF THE INVENTION

The present invention relates to methods and compositions for enhancing protein folding. In particular, the present invention relates to methods for directing recombinantly expressed proteins to protein folding chaperones.

### BACKGROUND OF THE INVENTION

Escherichia coli remains the organism of choice for synthesizing proteins for biochemical and structural studies due to the ease of growth and large number of available strains and expression vectors. It has become almost routine to clone genes from any organism into E. coli expression vectors where they can be transcribed and translated at high rates. Some vectors will even fuse the expressed protein to an affinity tag, making it easier to purify.

However, more often than not, a foreign protein will misfold and precipitate when synthesized in E. coli. Solubilizing and refolding precipitated proteins requires harsh treatments, and generally results in low yields and minimal or no activity of the refolded protein.

What is needed in the art are improved methods for folding exogenous proteins expressed in E. coli.

### SUMMARY OF THE INVENTION

The present invention relates to methods and compositions for enhancing protein folding. In particular, the present invention relates to methods for directing recombinantly expressed proteins to protein folding chaperones. Use of the compositions and methods of the present invention results in the expression of soluble proteins. Accordingly, in some embodiments, the present invention provides compositions, vectors, kits, and methods of using the compositions, kits, and vectors for expressing, purifying, and analyzing heterologous proteins of interest.

For example, in some embodiments, the present invention provides a method, comprising providing a vector comprising a gene encoding a heterologous protein of interest fused to a gene encoding at least a portion of a T4 head protein; and introducing the protein into a cell under conditions such that the protein of interest folds into an soluble protein. In some embodiments, fusion of the T4 head protein sequence to the protein of interest enhances protein folding (e.g., as compared to fusion to non T4 head protein sequences). In some embodiments, the at least a portion of a T4 head protein comprises at least a portion of a gene encoding the amino acid sequence of SEQ ID NO: 1. In other embodiments, the at least a portion of a T4 head protein comprises a gene encoding a portion of T4 head protein wherein the GOL region (e.g., amino acids 80-135 of SEQ ID NO:1) has been removed. In still further embodiments, the T4 head protein sequence comprises a sequence that is at least 80%, preferably at least 90% and even more preferably at least 99% homologous to SEQ ID NO:1. In preferred embodiments, the cell is E. coli. In some embodiments, the vector further comprises a protease cleavage site inserted in between the gene encoding a protein of interest and the gene encoding at least a portion of a T4 head protein. In other embodiments, the vector further comprises a nucleic acid sequence encoding a protein purification tag (e.g., an affinity tag), wherein the nucleic acid sequence is fused to the gene encoding a protein of interest. In some embodiments, the method further comprises the step of determining the amount of soluble protein of interest. In other embodiments, the method further comprises the step of determining the activity of the protein of interest. In certain embodiments, determining the activity of the protein of interest comprises performing an enzyme activity assay. In still other embodiments, the method further comprises the step of performing a drug screening assay with the protein of interest, wherein the drug screening assay comprises exposing the protein of interest to a test compound and determining the activity of the protein of interest in the presence and absence of the test compound.

The present invention further provides a kit comprising a vector comprising a gene encoding at least a portion of a T4 head protein fused to a site for inserting a heterologous gene of interest, wherein the gene of interest is fused to the at least a portion of a T4 head protein. In some embodiments, the at least a portion of a T4 head protein comprises at least a portion of a gene encoding the amino acid sequence of SEQ ID NO:1. In other embodiments, the at least a portion of a T4 head protein comprises a gene encoding a portion of T4 head protein wherein the GOL region (e.g., amino acids 80-135of SEQ ID NO:1) has been removed. In some embodiments, the vector further comprises a protease cleavage site inserted in between the gene encoding a protein of interest and the gene encoding at least a portion of a T4 head protein. In other embodiments, the vector further comprises a nucleic acid sequence encoding a protein purification tag (e.g., an affinity tag), wherein the nucleic acid sequence is fused to the gene encoding a protein of interest.

The present invention additionally provides a fusion protein comprising a protein of interest fused to at least a portion of a T4 head protein. In other embodiments, the present invention provides vector comprising a gene encoding a protein of interest fused to a gene encoding at least a portion of a T4 head protein.

### DESCRIPTION OF THE FIGURES

FIG. 1 shows Western blots of SDS-PAGE of fractions of lysed E. coli W31101acIq cells containing plasmids expressing each of the T4 fusion proteins. Cells were grown at 30° C. (FIG. 1A) and 42° C. (FIG. 1B).

FIG. 2 shows the relative fluorescence of the supernatants of extracts of cells shown in FIG. 1. Cells were grown at 30° C. (FIG. 2A) and 42° C. (FIG. 2B).

FIG. 3 shows colony growth and fluorescence on plates of E. coli W31101acIq cells expressing fusions of the head protein sequences to GFPmut2.

FIG. 4 shows colony growth of E. coli JM83 cells transformed by plasmids expressing fusions of the N terminus of the T4 head protein to a truncated β-gal protein.

FIG. 5 shows growth of E. coli W31101acIq cells expressing a fusion of sequences from the N terminus of the T4 head protein to the mutated form of GFPmut2with the mutations F8S and I161 S.

FIG. 6 shows colony growth and fluorescence of E. coli W31101acIq transformed by plasmids expressing fusions of the N terminus of the T4 head protein to the doubly mutated derivative of GFP-mut2, GFPmut2F8S,I 161 S.

FIG. 7 shows a Western blot of extracts of the cells in FIG. 6 expressing a fusion of the T4 head protein sequences to a mutant form of GFPmut2 with the changes F8S and I161S (2E,2S, and 2P).

FIG. 8 shows the amino acid sequence of amino acids 1- 176 of the T4 head protein (SEQ ID NO:1).

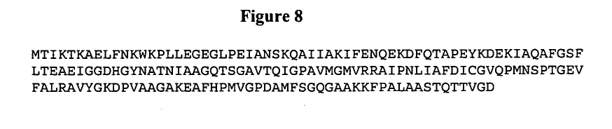

### DEFINITIONS

To facilitate an understanding of the present invention, a number of terms and phrases are defined below:

As used herein, the term “host cell” refers to any cell (e.g., bacterial, mammalian cells, avian cells, amphibian cells, plant cells, fish cells, and insect cells), whether located in vitro or in vivo. In preferred embodiments of the present invention the host cell is E. coli.

As used herein, the term “vector” refers to any genetic element, such as a plasmid, phage, transposon, cosmid, chromosome, virus, virion, etc., which is capable of replication when associated with the proper control elements within an appropriate host cell. Thus, the term includes cloning and expression vehicles, as well as viral vectors.

The term “nucleotide sequence of interest” refers to any nucleotide sequence (e.g., RNA or DNA), the manipulation of which may be deemed desirable for any reason (e.g., treat disease, confer improved qualities, expression of a protein of interest in a host cell, expression of a ribozyme, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

As used herein, the term “protein of interest” refers to a protein encoded by a nucleic acid of interest.

As used herein, the term “exogenous gene” refers to a gene that is not naturally present in a host organism or cell, or is artificially introduced into a host organism or cell.

As used herein, the terms immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F (ab′)₂ fragments, and Fab expression libraries; and single chain antibodies.

The term “epitope” as used herein refers to that portion of an antigen that makes contact with a particular immunoglobulin.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an two binding partners means that the interaction is dependent upon the presence of a particular structure (e.g., region) on the proteins; in other words the binding partners are recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an two binding partners refer to an interaction that is not dependent on the presence of a particular structure (e.g., the protein is binding to proteins in general rather that a particular structure such as an epitope).

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The RNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule increase the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample. “Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA or protein to indicate a level of expression approximately 2-fold higher (or greater) than that observed in a given tissue in a control. Levels of mRNA or protein are measured using any of a number of techniques known to those skilled in the art including, but not limited to Northern blot analysis and the quantitative immunofluorescence technique of the present invention (See e.g., Example 3). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthineguanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp.16.9-16.15.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reactions that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates to methods and compositions for enhancing protein folding. In particular, the present invention relates to methods for directing recombinantly expressed proteins to protein folding chaperones.

Manipulations of the protein chaperones offer a way to enhance the folding of foreign proteins in E. coli. There are three general types of protein chaperones in E. coli: DnaK, trigger factor and GroEL (for a review see Bakau and Horwich, Cell 92 (1998), pp. 351-366). The DnaK type of chaperone, called Hsp70 in eukaryotes, binds to the hydrophobic regions of proteins and prevents their premature association. Trigger factor works in a similar way (Teter et al., Cell 97 (1999), pp. 755-765; Deuerling et al., Nature 400 (1999), pp. 693-696) but is more closely associated with the exit pore of the ribosome. The other types of chaperones, represented by GroEL (Hsp60) in E. coli, are usually called chaperonins, to reflect their very different mode of action. They essentially act as a deus ex machina in the folding of proteins, taking up a misfolded protein and promoting its proper folding.

The GroEL chaperonin of E. coli was the first chaperonin discovered and has served as the prototype for the structure and function of the chaperonins (for a review see Sigler et al. Ann. Rev. Biochem. 67 (1998), pp. 581-608). Fourteen identical polypeptide products of the groEL gene form two large cylindrical chambers stacked back to back (Braig et al., Nature 371 (1994), pp. 578-586). The hydrophobic regions of an unfolded polypeptide bind to the relatively hydrophobic apical region of one of the chambers, the cis-chamber, and the polypeptide is taken up by the chamber. A cap, itself a heptamer formed of seven polypeptides encoded by the groES gene (Hunt et al., Nature 379 (1996), pp. 37-45.), is then put on the cis-chamber. The binding of the cochaperonin causes the cis-chamber to undergo a large conformational change, driving the hydrophobic regions of the protein internally and helping refold the protein (Wang and Boisvert, J. Mol. Biol. 327 (2003), pp. 843-855). Seven ATPs then bind to the other chamber, the trans-chamber. The seven ATPs on the cis-chamber are then cleaved to ADP, and the GroES cap is displaced, releasing the folded protein. The trans-chamber then becomes the cis-chamber, leading to a type of “two stroke” engine, with the folding role alternating between the two stacked chambers. The chaperonins are universal but exist in two general types: the Group I chaperonins represented by GroEL are found only in eubacteria and in the mitochondria and chloroplasts of eukaryotes, while the Group II chaperonins are found in archaea and the cytoplasm of eukaryotes. The two groups share a common overall structure but have little sequence homology and vary in the number of polypeptide subunits making up each of the chambers. While the Group I chaperonins have been thought to help a variety of proteins fold, including many denatured by heat or other stress conditions, the Group II chaperonins, at least those in the cytoplasm of eukaryotes, have been thought to be more dedicated to the folding and assembly of particular proteins such as actin or tubulin (Gao et al., Cell 69 (1992), pp. 1043-1050; Dunn et al., J. Struct. Biol. 135 (2001), pp. 176-184).

As many as 10% of all E. coli proteins bind to GroEL as they are being synthesized (Houry et al., Nature 402 (1999), pp. 147-154), but it is not known how many of these proteins actually depend upon GroEL for their normal folding or what features they share. Nevertheless, the folding of some essential protein or proteins of E. coli does depend upon GroEL under any conditions, since groEL is an essential gene at any temperature (Fayet et al., J. Bacteriol. 171 (1989), pp. 1379-1385). There is a strong motivation for finding GroEL-dependent proteins, since they offer clues as to what is required to direct proteins to GroEL chambers and help them fold.

The only protein known to require the GroEL chaperonin for its folding is the major head protein of T4 bacteriophage. The groEL gene was first found by mutations that prevent T4 and λ development (for a review see Ang et al., Ann. Rev. Genet. 34 (2000), pp. 439-456). The block in T4 development imposed by these groEL mutations can be partially bypassed by some mutations in the head protein gene of T4 (gene 23), suggesting that the head protein of the phage is the only essential T4 phage protein that absolutely requires GroEL for its folding. However, rather than use the host-encoded cochaperonin cap, GroES, the T4 head protein requires its own T4-encoded cochaperonin cap, Gp31, which shares no sequence homology with GroES but is structurally and functionally very similar (van der Vies et al., Nature 368 (1994), pp. 654-658; Hunt et al., Cell 90 (1997), pp. 361-371). Those groEL mutations that prevent T4 development do so mostly by weakening the binding of the Gp31 cap to the GroEL chamber (Klein and Georgopoulos, Genetics 158 (2001), pp. 507-517).

Not only does the head protein of T4 require GroEL for its folding, but a simple calculation suggests that it apparently is able to “commandeer” almost all of the cellular GroEL for this purpose. There are almost 1000 copies of the head protein in each T4 head and more than 100 phage heads are made per infected cell in a period of only 20-30 minutes later in infection. Therefore, during this time, more than 3000 head protein molecules are being pumped through GroEL chambers per minute in each infected cell. Based on estimates of the amount of GroEL polypeptide per cell at 37° C. (Neidhardt et al., Ann. Rev. Genet. 18 (1984), pp. 295-329), an E. coli cell contains only about 700 decatetrameric GroEL structures at 37° C. and even fewer at lower temperatures. Since T4 shuts off all host protein translation almost immediately after infection, it does not have the option of inducing more GroEL protein to meet its needs. Therefore, four to five head proteins must pass through each GroEL structure per minute, in a process that takes as long as 10 to 15 seconds for each folded protein (Sigler et al., Ann. Rev. Biochem. 67 (1998), pp. 581-608.). This leaves very little GroEL for other purposes. The present invention is not limited to a particular mechanism. Indeed, an understanding of the mechanism is not necessary to practice the present invention. Nonetheless, it is contemplated that the efficiency with which the T4 head protein uses GroEL suggests that it might contain sequences or structures that bind unusually strongly to GroEL, allowing it to outcompete other proteins for GroEL binding.

Experiments conducted during the course of development of the present invention discovered that a short region about 100 amino acid residues in from the N terminus of the T4 head protein, called the Gol region, binds to translation elongation factor Tu (EF-Tu) as the head protein emerges from the ribosome (Bergsland et al., J. Mol. Biol. 213 (1990), pp. 477-494; Yu and Snyder, Proc. Natl Acad. Sci. USA 91 (1994), pp. 802-806; Georgiou et al., Proc. Natl Acad. Sci. USA 95 (1998), pp. 2891-2895; Bingham et al., J. Biol. Chem. 275 (2000), pp. 23219-23226.). This binding induces a pause in translation of the head protein in the vicinity of the Gol region (Snyder et al., J. Mol. Biol. 334 (2003), pp.349-361). It is contemplated that the pause occurs because EF-Tu in a complex with the Gol region can enter the A site of the ribosome from which that Gol region has just emerged, occluding aminoacylated tRNA from that site. It is further contemplated that the purpose of this pause was to stop translation of that head protein until GroEL could bind to sequences in the head protein upstream of the Gol region. Translation then continues, forcing cotranslation of the head protein with its insertion into a GroEL chamber, and preventing premature synthesis and precipitation of the head protein. One feature of this model is that the EF-Tu-Gol region complex, bound to the A site of the stalled ribosome, also occludes tmRNA from that site, thereby preventing premature termination of translation and degradation of the head protein.

This model predicts the existence of sequences or structures in the head protein sequences upstream of the Gol region that bind to GroEL, relieving the stalled complexes. The present invention is not limited to a particular mechanism. Indeed, an understanding of the mechanism is not-necessary to practice the present invention. Nonetheless, it is contemplated that if these sequences bind strongly to GroEL and thereby direct the emerging head protein to a GroEL chamber, then the same sequences, when fused to a reporter protein, also direct the reporter protein in the fusion to GroEL, and promote its folding. Experiments conducted during the course of development of the present invention provided evidence consistent with this prediction. The green fluorescent protein (GFP) can fold at higher temperatures when it is fused to the N terminus of the T4 head protein than if it is synthesized autonomously or fused to other protein sequences. It is contemplated that the head protein sequences in the fusions compete so effectively for GroEL that they deplete the cell of GroEL needed for other essential functions. Accordingly, in some embodiments, the present invention provides methods of targeting proteins to the GroEL chaperonin and thereby promoting their proper folding. The methods and compositions of the present invention are suitable for use in aiding the folding of any foreign or exogenous protein expressed in E. coli.

The efficiency with which the T4 head protein cycles through GroEL later in phage infection suggests that the head protein contains sequences that direct it strongly to GroEL. Experiments conducted during the course of development of the present invention demonstrated that fusing N-terminal sequences from the T4 head protein to GFP helps the GFP in the fusion fold, even at temperatures higher than those at which GFP can normally fold. Thus, the sequences within the head protein that normally bind strongly to GroEL reside in these N-terminal sequences, where they can also direct the fusion protein including the GFP protein to GroEL. The GFP protein in the fusion then folds more effectively within a GroEL chamber than it can fold autonomously.

The present invention is not limited to a particular mechanism. Indeed, an understanding of the mechanism is not necessary to practice the present invention. Nonetheless, there are several mechanisms by which the head protein sequences might enhance the folding of proteins. However, the toxicity of some of the fusions implicates GroEL in the enhanced folding. The evidence suggests that the toxicity is due to the head protein sequences competing so effectively for GroEL that they deplete the cells of the GroEL needed for other cellular processes. The fusion proteins are toxic even when synthesized in only moderate amounts, suggesting that they are having a specific effect on the cell, rather than, for example, overwhelming the protein synthetic capacity of the cell. Furthermore, conditions that might be expected to increase the availability of GroEL in the cell, for example higher temperatures, can relieve the toxicity, as though, by providing enough GroEL to go around, they relieve the competition for GroEL. The fact that some mutations in the groEL gene can also relieve the toxicity further implicates GroEL. At least one of the normal cellular proteins that depends upon GroEL for its functioning, and is therefore malfunctioning as a result of GroEL deprivation, is required to maintain the integrity of the cell since proteins leak from the cell after induction of the fusion protein.

While the toxicity of the fusion proteins is due to the head protein sequences in the fusion, replacing gfp in the fusion with other reporter genes affects the severity of the toxicity. The length of the reporter gene has an effect on folding, as do mutations that promote the crosslinking of the reporter gene in the fusion. The reporter gene in the fusion does matter even if it is the head protein sequences that cause the fusion protein to bind strongly to GroEL. The toxicity may be affected by many factors, including whether the fusion protein can fit into a GroEL chamber, whether it can jam GroEL chambers when it is taken up, and whether it precipitates irreversibly before it can be taken up.

The sequences within the N terminus of the T4 head protein are responsible for the strong binding to GroEL lie within the first 176 amino acid residues of the protein, exclusive of the Gol region.

In experiments conducted during the course of development of the present invention the presence or absence of the Gol region in the upstream T4 head sequences did not effect folding. The Gol region binds to EF-Tu and promotes a pause in translation (Snyder et al., J. Mol. Biol. 334 (2003), pp. 349-361). It is contemplated that some proteins, including the head protein of the phage, might irreversibly precipitate in the absence of such Gol site-enforced cotranslation. The T4 head protein is well known to irreversibly precipitate if it is synthesized in its entirety without being taken up by GroEL (Coppo et al., J. Mol. Biol. 96 (1973), pp. 61-87). It is contemplated that other proteins, including GFP, do not require cotranslation and are taken up and refolded even after they are synthesized. In some embodiments, other proteins, such as the phage-encoded Gp31 cochaperonin, that are present in the infected cell participate with the Gol region in head protein folding.

Chaperonin-induced folding is widely distributed, and, except for the specificity in which proteins are taken up, the general process that is used does not differ significantly between different types of proteins.

Accordingly, in some embodiments, the present invention provides methods and compositions for enhancing protein folding by fusing a gene encoding a protein of interest to a gene encoding a T4 head protein. The below discussion provides a non-limiting discussion of exemplary compositions and methods of the present invention. One skilled in the art recognizes that additional embodiments are within the scope of the invention.

I. Expression and Folding of Fusion Proteins

As described above, in some embodiments, the present invention provides methods and compositions for expressing fusion proteins comprising a gene encoding a protein of interest fused to a gene encoding a T4 head protein. The methods and compositions of the present invention are suitable for expressing any exogenous protein of interest in E. coli, including, but not limited to proteins of viral, bacterial, or eukaryotic origin. Proteins that are normally cytoplasmic or monomeric are particularly well suited for use with the methods of the present invention.

By way of example, the below description focuses on expression of heterologous proteins fused to T4 head protein sequences in E. coli. However, the present invention is not limited to the use of T4 head protein sequences in E. Coli. Additional targeting proteins and host cells are specifically contemplated by the present invention.

A. Vectors

In some embodiments, the present invention provides vectors for use in expressing a gene of interest fused to a gene encoding at least a portion of the T4 head protein. The present invention is not limited to a particular portion of the T4 head protein. In some embodiments, the entire T4 head protein is included. In other embodiments, N-terminal portions of the T4 head protein are utilized (e.g., at least 100, preferably at least 150 and even more preferably at least the first 170 amino acid). In some particularly preferred embodiments, amino acids 1- 170 of the T4 head protein (described by SEQ ID NO:1) are utilized. In some embodiments, the GOL region (approximately amino acids 80-150 of SEQ ID NO:1) are removed.

The present invention further contemplates variants of SEQ ID NO:1 that may or may not be functional and share some homology with SEQ ID NO:1. Preferred variants have the activity of interacting with GroEL and are at least 60%, preferably at least 70%, even more preferably at least 80%, still more preferably at least 90%, yet more preferably at least 95% and most preferably at least 99% homologous to SEQ ID NO:1. Particularly preferred T4 head protein sequences enhance the folding of a fused protein of interest relative to the folding in the absence of fusion to another protein or as compared to proteins fused to non-T4 head protein sequences.

The present invention is also not limited to the T4 head protein. Other proteins that direct proteins of interest to chaperones may be utilized. For example, in some embodiments, FtsZ proteins are utilized to direct proteins of interest to CTT chaperonins in eukaryotic cells.

In some embodiments, the protein of interest is fused to the T4 head protein using a linker. In some embodiments, the linker comprises a site for protease binding (e.g., thrombin or other protease). The T4 sequences can then be easily cut off the protein after it has folded. In other embodiments, the linker comprises an intein.

In some embodiments, vectors further comprise a gene encoding a protein purification tag fused to the gene encoding a protein of interest. Protein purification tags (e.g., affinity tags) are useful in the purification of expressed proteins. Examples of protein purification tags include, but are not limited to, polyhisitidine tags.

In some embodiments, the concentration of cellular GroEL is increased (e.g., by overexpressing recombinant GroEL) to increase the capacity of the folding system. The present invention is not limited to a particular mechanism. Indeed, an understanding of the mechanism is not necessary to practice the present invention. Nonetheless, it is contemplated that the presence of additional GroEL will enhance the protein folding abilities of cell expressing proteins of interest fused to T4 head proteins.

In preferred embodiments, vectors are suitable for use in E. coli. In some embodiments of the present invention, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). It is contemplated that any vector may be used as long as it is replicable and viable in the host.

In preferred embodiments of the present invention, the appropriate DNA sequence is inserted into the vector using any of a variety of procedures. In general, the DNA sequence is inserted into an appropriate restriction endonuclease site(s) by procedures known in the art.

Large numbers of suitable vectors are known to those of skill in the art, and are commercially available. Such vectors include, but are not limited to, the following vectors: 1) Bacterial—pQE70, pQE60, pQE-9 (Qiagen), pBS, pD10, phagescript, psiXI174, pbluescript SK, pBSKS, pNH8A, pNH16a, pNH18A, pNH46A (Stratagene); ptrc99a, pKK223-3, pKK233-3, pDR540, pRIT5 (Pharmacia); 2) Eukaryotic—pWLNEO, pSV2CAT, pOG44, PXT1, pSG (Stratagene) pSVK3, pBPV, pMSG, pSVL (Pharmacia); and 3) Baculovirus—pPbac and pMbac (Stratagene). Any other plasmid or vector may be used as long as they are replicable and viable in the host. In some preferred embodiments of the present invention, mammalian expression vectors comprise an origin of replication, a suitable promoter and enhancer, and also any necessary ribosome binding sites, polyadenylation sites, splice donor and acceptor sites, transcriptional termination sequences, and 5′ flanking non-transcribed sequences. In other embodiments, DNA sequences derived from the SV40 splice, and polyadenylation sites may be used to provide the required non-transcribed genetic elements.

In certain embodiments of the present invention, the DNA sequence in the expression vector is operatively linked to an appropriate expression control sequence(s) (promoter) to direct mRNA synthesis. Promoters useful in the present invention include, but are not limited to, the LTR or SV40 promoter, the E. coli lac or trp, the phage lambda P_{L} and P_{R}, T3 and T7 promoters, and the cytomegalovirus (CMV) immediate early, herpes simplex virus (HSV) thymidine kinase, and mouse metallothionein-I promoters and other promoters known to control expression of gene in prokaryotic or eukaryotic cells or their viruses. In other embodiments of the present invention, recombinant expression vectors include origins of replication and selectable markers permitting transformation of the host cell (e.g., dihydrofolate reductase or neomycin resistance for eukaryotic cell culture, or tetracycline or ampicillin resistance in E. coli).

In some embodiments of the present invention, transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Enhancers useful in the present invention include, but are not limited to, the SV40 enhancer on the late side of the replication origin bp 100 to 270, a cytomegalovirus early promoter enhancer, the polyoma enhancer on the late side of the replication origin, and adenovirus enhancers.

In other embodiments, the expression vector also contains a ribosome-binding site for translation initiation and a transcription terminator. In still other embodiments of the present invention, the vector may also include appropriate sequences for amplifying expression.

B. Host Cells

In preferred embodiments, E. coli is utilized as a host cell. However, the present invention is not limited to use in E. coli. In other embodiments of the present invention, the host cell is a lower eukaryotic cell (e.g., a yeast or mammalian cell). In still other embodiments of the present invention, the host cell is a prokaryotic cell (e.g., a bacterial cell). Specific examples of host cells include, but are not limited to, Escherichia coli, Salmonella typhimurium, Bacillus subtilis, and various species within the genera Pseudomonas, Streptomyces, and Staphylococcus, as well as Saccharomycees cerivisiae, Schizosaccharomycees pombe, Drosophila S2 cells, Spodoptera Sf9 cells, Chinese hamster ovary (CHO) cells, COS-7 lines of monkey kidney fibroblasts, (Gluzman, Cell 23:175 [1981]), C127, 3T3, 293, 293T, HeLa and BHK cell lines.

The constructs in host cells can be used in a conventional manner to produce the fusion proteins encoded by the recombinant sequence. In some embodiments, introduction of the construct into the host cell can be accomplished by calcium phosphate transfection, DEAE-Dextran mediated transfection, or electroporation (See e.g., Davis et al., Basic Methods in Molecular Biology, [1986]). Alternatively, in some embodiments of the present invention, the fusion polypeptides of the invention can be synthetically produced by conventional peptide synthesizers.

Fusion proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., [1989].

In some embodiments of the present invention, following transformation of a suitable host strain and growth of the host strain to an appropriate cell density, the selected promoter is induced by appropriate means (e.g., temperature shift or chemical induction) and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

C. In Vitro Expression and Folding

The present invention is not limited to expression and folding of proteins in host cells. In some embodiments, fusion protein of a protein of interest to at least a portion of a T4 head protein is expressed in vitro. In some embodiments, GroEL is expressed in the same vessel and the protein of interest is folded in vitro. In other embodiments, GroEL is expressed and purified separately and combined with fusion protein for the folding step.

D. Purification of Proteins

The present invention also provides methods for recovering and purifying proteins of interest from recombinant cell cultures including, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. In other embodiments of the present invention, protein-refolding steps can be used as necessary, in completing configuration of the mature protein. In still other embodiments of the present invention, high performance liquid chromatography (HPLC) can be employed for final purification steps.

The present invention further provides polynucleotides having the coding sequence of a protein of interest fused in frame to a marker sequence that allows for purification of the polypeptide of the present invention. A non-limiting example of a marker sequence is a hexahistidine tag which may be supplied by a vector, preferably a pQE-9 vector, which provides for purification of the polypeptide fused to the marker in the case of a bacterial host, or, for example, the marker sequence may be a hemagglutinin (HA) tag when a mammalian host (e.g., COS-7 cells) is used. The HA tag corresponds to an epitope derived from the influenza hemagglutinin protein (Wilson et al., Cell, 37:767 [1984]).

E. Kits

In some embodiments, the present invention provides kits for expressing and folding proteins of interest. In some embodiments, the kits contain at least one vector encoding at least a portion of a T4 head protein fused to a cloning site for inserting a gene encoding a protein of interest. In some further embodiments, the vector includes a linker (e.g., comprising a recognition site for a protease) located in between the gene encoding at least a portion of a T4 head protein and the site for inserting a gene encoding a protein of interest. In other embodiments, the vector further includes a nucleic acid sequence encoding a protein purification tag (e.g., an affinity tag) such that the tag will be fused to the protein of interest.

In preferred embodiments, the kits contain all of the components necessary to clone a gene encoding a protein of interest into the vector, as well as control plasmids and any buffers needed for expression of a protein of interest. In some embodiments, the kit comprises instructions for using the kit to clone and express a protein of interest.

In yet other embodiments, the kit further comprises components and instructions for analyzing expressed and/or purified proteins. For example, in some embodiments, the kits include components for performing protein or enzyme activity assays.

II. Applications

In some embodiments, the present invention provides compositions and methods for expressing soluble exogenous proteins in E. coli. The expressed and optionally purified proteins find use in any application in which it is desirable to utilize soluble or active protein.

In some embodiments, proteins are used in basic research applications. For example, in some embodiments, proteins are purified and used in studies of protein structure. Many techniques for studying protein structure are known in the art and include, but are not limited to, X-ray crystallography, spectroscopy (e.g., UV or Infared spectroscopy), electrophoresis and mass spectrometry.

In other embodiments, proteins are utilized in studies of protein function. Such studies include, but are not limited to, assays of enzymatic activity and interaction with a ligand or substrate or other molecule. Methods for performing such assays are known to one of skill in the art.

In yet other embodiments, expressed proteins are used in drug screening applications. In some embodiments, libraries of compounds are screened for their effect on the activity of purified proteins of interest.

In some preferred embodiments, high throughput drug screening assays are performed. Both in vitro assays utilizing purified proteins and cell culture assays where cells express recombinant proteins of interest are amenable to high throughput screening methods. In certain embodiments, multiplexed drug screening assays are utilized to screen multiple interactions simultaneously.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37:2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. NatI. Acad. Sci. 87:6378-6382 [1990]; Felici, J. Mol. Biol. 222:301 [1991]).

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); and ° C. (degrees Centigrade).

### Example 1

A. Methods

Plasmid and Strain Construction

The plasmid pGLZ30 was the starting point for the construction of fusion proteins (Maldonado et al., J. Bacteriol. 180 (1998), pp. 1822-1830). It has the entire lacZ gene of E. coli transcribed from the tac promoter and a HindIII site just downstream of a Shine and Dalgarno sequence on the plasmid into which genes to be translated can be cloned. The construction of plasmids, pGL231-176-lacZ and pGL231-176Δgol-lacZ, in which the coding sequence of the N terminus of the head protein including the AUG are fused to lacZ in this vector have been described (Snyder et al., J. Mol. Biol. 334 (2003), pp. 349-361) but renamed here. Briefly, PCR was used to amplify the coding sequence for the N terminus of the head protein using primers that introduce a HindIII site just upstream of the AUG of the head protein gene (gene 23) and an ApaI site just downstream of the region to be fused. This amplified fragment was then cloned between the HindIII and ApaI sites on pGLZ30 so that varying lengths of the N terminus of the head protein replaced the N terminus of β-galactosidase on the cloning vector. The Gol region extends from approximately A94 to G122 in the head protein (Bergsland et al., J. Mol. Biol. 213 (1990), pp. 477-494). To delete the Gol region from the plasmid, a PstI site was introduced by PCR around the codon for K136 in gene 23 cloned in a different vector that lacks a Pstl site of its own, and the plasmid was cut with PstI and religated. This removes the coding region from the natural PstI site in gene 23 from approximately A80 to approximately K136 and thus removes the sequence encoding the Gol region and a few amino acid residues on each side of it. The N-terminal coding sequence of the head protein was then PCR-amplified from this plasmid and recloned into pGLZ30 as above. To truncate the β-gal in the fusions to make pGL231-176-lacZΔB and pGL231-176Δgol-lacZΔB, the plasmids pGL231-176-lacZ and pGL231-176Δgol-lacZ were cut at the unique Bc1I and BamHI sites in the lacZ gene and religated. This also removes the UAA stop codon at the end of the lacZ gene and adds about 150 amino acid residues to the end of the protein. The plasmid fusions to the gfpmut2 gene, pGL231-176-gfp-mut2 and pGL231-176Δgol-gfp-mut2 were made by substituting the gfp gene for the lacZ gene in the above plasmids. PCR was used to amplify the gfp gene from plasmid pKL14724 and introduce an ApaI site upstream of the coding sequence for K3 in gfpmut2 and a BamH1 site just downstream of the UAG stop codon. The gfp gene in this plasmid is not the original gfpmut2, but has three other mutations, S65A, V68L and S72A, which help it fold better in E. coli. This amplified fragment was cloned into pGL231-176-lacZ cut with ApaI and BamHI, and the cloned gfp gene was sequenced to be certain no additional mutations had been introduced by the PCR amplification. Once an unmutated gfpmut2 gene clone had been identified, this gene was cut out of the plasmid and used to make the other plasmids. The plasmid pGL-gfpmut2 was made by cloning this gfpmut2 gene into just the pGLZ30 cloning vector cut with ApaI and BamHI so that the GFP protein was fused to the N terminus of β-gal encoded by the cloning vector. The construction of pGL2380-135-gfp-mut2, in which just the Gol region in the T4 head protein is fused to GFP, was made by cloning this amplified gfp gene into pGLG, which has been described (Snyder et al., J. Mol. Biol. 334 (2003), pp. 349-361).

The GroEL44 and GroEL673 mutations have been described (Zeilstra-Ryalls et al., J. Bacteriol. 175 (1993), pp.1 134-1143). They were transduced by P1 into E. coli JM8331 and other strains by selecting the tetracycline resistance (Tcr) on a nearby Tn10 transposon, which is not cotransducible with groEL. The Tcr transductants into which the groEL mutation had been cotransduced were identified because they could not plate wild-type T4 phage, but could plate Gp23 and Gp31 bypass mutants. A Tcr transductant that was still wild-type for groEL was retained for use as a control. The E. coli strain W3110lacIq has been described (Bergsland et al., J. Mol. Biol. 213 (1990), pp. 477-494). It has a very strong laclq gene in its chromosome and was made lacZ- by a Tn5 insertion mutation.

Growth of Cells and Preparation of Extracts

To prepare extracts for Western blot analysis and fluorescence measurements, transformants of cells were grown overnight without shaking in LB medium plus ampicillin and diluted about 1:20 into 50 ml of fresh medium to begin growth. They were grown to an A625 nm of 0.2 before IPTG was added to 1 mM. Two hours later, the cells were centrifuged and resuspended in 5 ml of buffer A (10 mM Tris-HCl (pH 7.5), 10 mM MgCl2, 25 mM NaCl). They were then lysed by sonication and centrifuged for one hour at 27,000 g to pellet any precipitated protein. The gently washed pellet was resuspended in 5 ml of buffer A by a brief burst of sonication, and aliquots of the original lysate, supernatant and pellet were added to an equal volume of 2× cracking buffer (0.125 M Tris-HCl (pH 6.8), 4% (w/v) SDS, 20% (v/v) glycerol, 10 mM fresh dithiothreitol) and boiled two minutes. Aliquots were then subjected to 10% (w/v) SDS-PAGE electrophoresis and electroblotted on nitrocellulose paper and probed with polyclonal rabbit anti-GFP antibody followed by anti-rabbit IgG-alkaline phosphatase secondary antibody (Promega, Madison, Wis.). The lysis technique was initially monitored by anti-β-gal monoclonal antibodies (Promega) directed against the chromosomally encoded β-gal protein, almost all of which remained in the supernatant. Fluorescence was measured in microtiter plates using a Perkin-Elmer LS50B Luminescence Spectrometer, with an excitation wavelength of 490 mv and an emission wavelength of 510 mp, with a 5 mv bandwidth. Background fluorescence of the supernatant of cells not containing a plasmid, but otherwise treated identically, were subtracted from each measurement.

B. Results

Use of GFP Fusions to Determine the Effect of T4 Head Protein Sequences on Protein Folding

The present invention is not limited to a particular mechanism. Indeed, an understanding of the mechanism is not necessary to practice the present invention. Nonetheless, it is contemplated that if sequences upstream of the Gol region in the T4 head protein help direct the head protein to GroEL, that fusing these sequences to a reporter gene product will direct the reporter gene product to GroEL and help it fold. The GFP from a jellyfish was used as a reporter protein to test this hypothesis. The GFP protein is often used to assay protein folding because fluorescence by GFP requires the formation of a cyclic chromophore involving three contiguous amino acids in the protein and the reactions to form the chromophore will only occur if the protein has first folded properly (Tsien, Ann. Rev. Biochem. 67 (1998), pp. 509-544). Another advantage is that GFP is known to fold when fused to many other protein sequences. Also, at only about 24 kDa, the GFP protein is relatively small and fits into a GroEL chamber, even when fused to the N terminus of the T4 head protein. Many derivatives of the original GFP protein, such as GFP-mut2, have been constructed which fold in E. coli (Cormack et al., Gene 173 (1996), pp. 33-38). However, this folding is temperature-sensitive, with poor folding at higher temperatures.

To determine the effect of being fused to head protein sequences on the folding of GFP, the plasmids pGL231-176-gfp-mut2 and pGL231-176Δgol-gfp-mut2, which express a fusion protein in which the first 176 amino acid residues of the head protein (gene 23) are fused to a further E. coli-tized version of GFPmut2 were used. The only difference between these two plasmids is that, in the second plasmid, a short in-frame deletion of amino acid residues 80-135 has removed the Gol region and a few surrounding amino acids from the T4 head protein sequences in the fusion protein. As controls, two other plasmids were constructed: pGL-gfpmut2, and pGL2380-135-gfp-mut2, which express a fusion protein that lacks the upstream T4 head protein sequences. In the first plasmid, pGL-gfpmut2, just the gfpmut2 gene has been cloned into the cloning vector, so that the GFP protein will be fused to just a few amino acid residues from the N terminus of β-gal encoded by the cloning vector; and in pGL2380-135-gfp-mut2, only the Gol region and a few surrounding amino acids from the T4 head protein are fused to GFP.

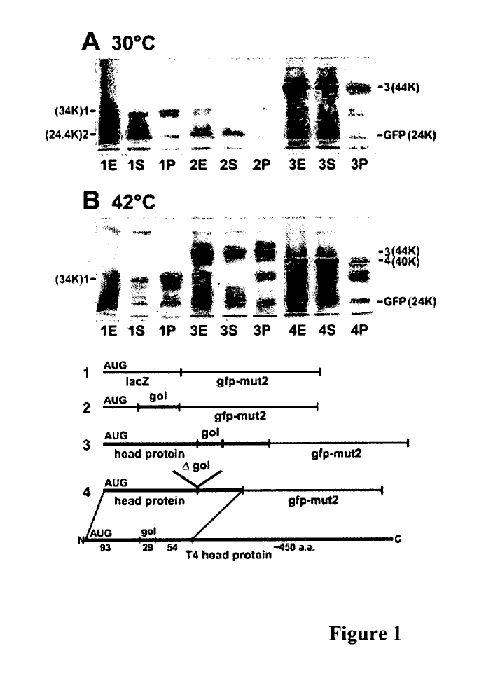

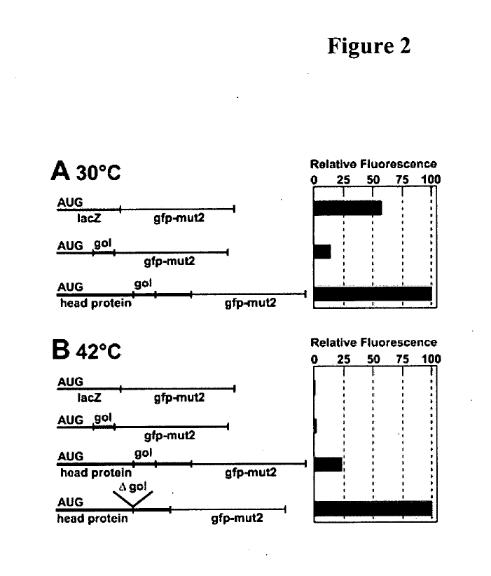

All four plasmids were used to transform E. coli W3110 lacIq, and the transformants were grown at 30° C. and induced with IPTG as described above. To determine the amount of each fusion protein made and its solubility, the cells were harvested, disrupted by sonication, and centrifuged to pellet any precipitated proteins. The pellets were resuspended, and the original lysate, supernatant, and pellet were subjected to SDS-PAGE and electroblotted for Western blot analysis using anti-GFP antibodies (FIG. 1(A)). In all cases, the GFP fusion proteins remained mostly in the supernatant, indicating that they had folded properly. When direct fluorescence measurements were done on the supernatants of the lysed cells (FIG. 2(A)), they were commensurate with the amount of the GFP fusion protein in the supernatants, based on the Western blots. This indicates that the GFP part of the fusion proteins remaining in the supernatant had folded properly, allowing the chromophore to form. These results were therefore in agreement with the anticipated result that GFPmut2 synthesized at lower temperatures can fold quite well in E. Coli, even if fused to other protein sequences, including those from the N terminus of the T4 head protein.

It was next determined whether the N-terminal T4 head protein sequences can help GFPmut2 fold at higher temperatures. Therefore, cells were grown at 30° C., as before, but shifted them to 42° C. before adding IPTG to induce synthesis of the fusion proteins. The cells were then lysed and the solubility of the fusion proteins and their fluorescence measured as before (FIG. 1 and FIG. 2). The folding of GFP strongly depended upon whether or not it was fused to the N-terminal T4 head protein sequences. The GFP protein fused to the N terminus of β-gal mostly precipitated (FIG. 1(B) lanes 1E, 1S and 1P), and the little that remained in the supernatant fluoresced almost not at all (FIG. 2(B)). GFPmut2 fused to just the Gol region of the major head protein also folded poorly and mostly precipitated and fluoresced almost not at all (FIG. 2(B)). GFPmut2 fused to the first 176 amino acid residues of the T4 head protein remained largely soluble (FIG. 1(B), lanes 3E,3S and 3P) and fluoresced very actively (FIG. 2(B)), indicating that much of it had folded properly even at 42° C. In cells transformed by the plasmid, pGL231-176Δgol-gfp-mut2, which expresses a fusion protein in which the first 176 amino acid residues of the head protein are fused to GFPmut2 but with the Gol region deleted from the head protein sequences, almost all of the fusion protein remained in the supernatant (FIG. 1(B), lanes 4E, 4S and 4P) and the GFP protein in the fusion fluoresced very actively (FIG. 2(B)). The GFP portion of this fusion folded and fluoresced as well when synthesized at 42° C. as it did when synthesized at 30° C. Thus, sequences in the N terminus of the T4 head protein, exclusive of the Gol region, contribute to the folding and consequent fluorescence of GFP protein to which they are fused. This is consistent with the model that the N terminus of the T4 head protein contains sequences that direct the protein strongly to GroEL and that the improved folding is occurring within GroEL chambers.

The Western blots in FIG. 1 show a difference in the amounts of the fusion proteins made. More fusion protein was made when GFP was fused to the N terminus of the head protein (lane 3E) than when the GFP was fused to the N terminus of β-gal encoded in the cloning vector (lane 1E) or to just the Gol region of the head protein (lane 2E). Some of these differences are attributable to variations in the strengths of their respective translation initiation regions (TIRs), even though they are all translated from the same S-D sequence on the plasmid. The TIR for the T4 head protein is very strong and differences downstream of the initiator codon can also affect the strength of a TIR. The presence of the Gol region also generally reduces the amount of fusion protein made, consistent with previously reported results that EF-Tu binding to the Gol region causes a pause in translation (Snyder et al., J. Mol. Biol. 334 (2003), pp. 349-361). Differences in the relative stabilities of the fusion proteins may also contribute.

Another feature of the Western blots in FIG. 1 is that some of the GFP in the supernatants ran as the original length GFP polypeptide, and not as the longer fusion polypeptide. It is contemplated that these smaller fragments are the products of proteolysis and not the result of premature termination of translation, since the antibody used to detect the fusion protein is directed against the GFP part of the fusion on the carboxyl end. This suggests that the unfolded nature of the N terminal β-gal or T4 head protein sequences make them a target for proteases, which leave the folded GFP portion of the fusion intact. This conclusion is supported by the observation that fusion protein in the pellet tends to be full length (compare for example FIG. 1(A), lanes 1S to lane 1P or FIG. 1(A), lane 3S to 3P), suggesting that the unfolded N-terminal sequences are made inaccessible to proteases if the fusion protein immediately precipitates after it is made.

The present invention is not limited to a particular mechanism. Indeed, an understanding of the mechanism is not necessary to practice the present invention. Nonetheless, is it contemplated that there are a number of ways being fused to the T4 head protein sequences could help the GFP protein fold at high temperatures. The below discussion is not intended to limit the invention to any particular mechanism, but rather to provides a discussion of exemplary mechanisms.

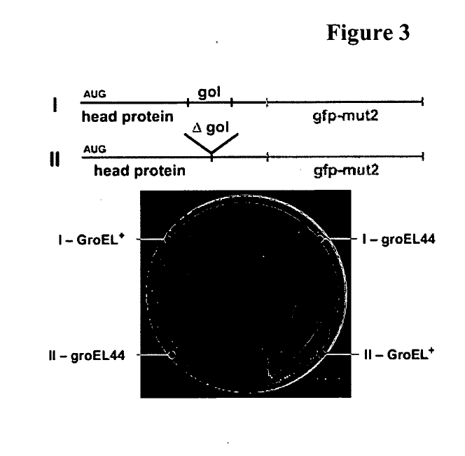

Further evidence supports the role of GroEL in the enhanced folding. Cells transformed by the plasmid pGL231-176Δgol-gfp-mut2, which expresses GFPmut2 fused to the first 176 amino acid residues of the head protein, but with the Gol region deleted, did not grow well when plated on selective plates containing the inducer IPTG at 30° C., and took two or more days of incubation to form even tiny colonies (FIG. 3 sector II-GroEL+). Cells transformed by any of the other plasmids formed colonies after only one day of incubation. Moreover, not only did the colonies of bacteria expressing this particular fusion to the head protein sequences take longer to appear, but when tiny colonies finally did appear, they fluoresced very bright green, much brighter than colonies of cells expressing the other fusions (FIG. 3, compare sector II GroEL+ to the other sectors).

Additional experiments revealed that the very bright fluorescence of colonies of bacteria expressing this fusion is a plate artifact and does not reflect the absolute amount of fluorescing GFP in the cells. The slow growth of the cells expressing this fusion and the bright green color of the colonies they formed were much less pronounced if the plate had been incubated at 37° C. or higher or if the cells had the groEL44 mutation in their chromosome (FIG. 3, sector II-groEL44). Western blots revealed that the amount of soluble fusion protein made under these conditions was not significantly different, nor was the fluorescence of the GFP in the fusion protein when measured directly in a fluorimeter. Thus, it is contemplated that expression of the fusion protein is causing a destabilization of the cellular membranes, allowing leakage of the fusion protein from some of the cells where it is more accessible to the excitation and or emitted light, and making the GFP in the fusion appear to be fluorescing very strongly.

The effects of the head protein sequences fused to GFP can by explained by binding of sequences within the N terminus of the T4 head protein to GroEL. It is contemplated that these sequences bind so strongly to GroEL that they allow the fusion protein to out-compete other cellular GroEL-dependent proteins for binding to GroEL, and commandeer almost all of the cellular GroEL. This competition would make the cell grow poorly by depriving it of the GroEL needed to fold its own GroEL-dependent proteins. If one or more of these out-competed GroEL-dependent E. Coli proteins are required for membrane stability, then depletion of available GroEL could cause the protein(s) to not fold properly, or not be inserted into the membranes, leading to the plate artifact described above. The ameliorating effect of high temperature can also be explained this way. Since GroEL is a heat shock protein, there is more GroEL in the cell to go around at higher temperatures, lessening the competition. There may also be more denatured proteins competing for the extra GroEL at the higher temperature, but the essential GroEL-dependent E. coli protein(s) may compete effectively with these denatured proteins for the available GroEL, even if they cannot compete effectively with the T4 head protein. Both mutant forms of GroEL, GroEL44 with the change E191G, and GroEL673 with the two changes G 173D and G337D (Zeilstra-Ryalls et al., J. Bacteriol. 175 (1993), pp.1 134-1143) are thought to interfere with T4 development by weakening the interaction with the Gp31 cochaperonin (Klein and Georgopoulos, Genetics 158 (2001), pp. 507-517). It is contemplated that the difference in the effects of the two GroEL mutations on the toxicity of the fusion protein are due to the groEL44 mutation changing another property of GroEL, for example, increasing the cellular levels of GroEL, or weakening the binding of the head protein sequences to GroEL, thereby lessening the competition. It is further contemplated that the fact that a groEL mutation can have an effect on the toxicity of the fusions implicates GroEL in the toxicity.

Fusions of the head protein sequences to other reporter genes have similar effects

It is contemplated that if the toxicity of the fusion protein is due to the head protein sequences in the fusion strongly directing the fuision protein to GroEL, then fusions of these same head protein sequences to other reporter proteins should also be toxic. To test this, the plasmid pGL231-176Δgol-lacZΔB, which is identical to pGL231-176Δgol-gfpmut2 except that a truncated form of the lacZ gene encoding β-galactosidase (β-gal) has replaced the gfpmut2 gene in the fusion was utilized. The β-gal reporter is very stable and folds to form dimers, even when fused to many other protein sequences. However, it is 114 kDa, much too long to fit into a GroEL chamber, which will only accommodate proteins of about 60 kDa or less. Longer fusions can be folded by GroEL but only on the outside of one of the chambers (Chaudhuri et al., Cell 107 (2001), pp. 235-246). Truncating β-gal lowers the size to only about 70 kDa, closer to the size that can be accommodated. By itself, the truncated β-gal protein does not have ,β-gal activity. However, if it is expressed in cells, such as E. coli JM83, that contain the carboxyl terminus of β-gal, the two parts of β-gal can complement each other to give active β-gal and the colonies will be blue on Xgal plates (α-complementation).

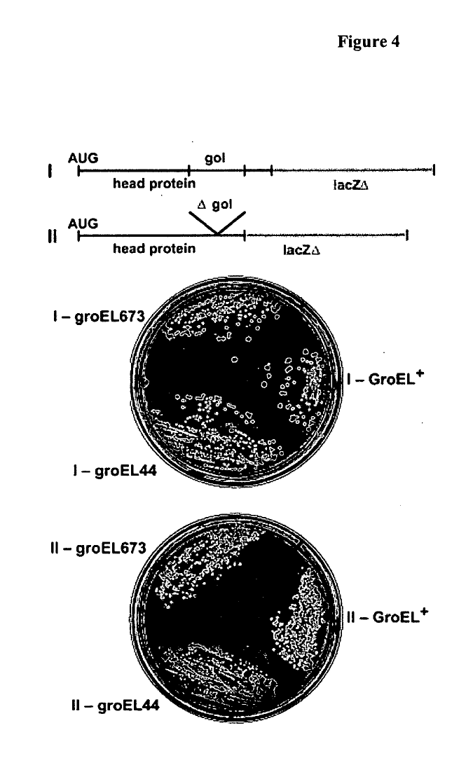

FIG. 4 shows the results when E. coli JM83 cells were transformed with the plasmid expressing this truncated fusion, and the transformation mix was spread on LB plates containing ampicillin, Xgal and IPTG and incubated at 30° C. In cells having the wild-type GroEL gene (sector II-GroEL+), large white colonies appeared after two or more days of incubation. These white colonies are mostly due the growth of “satellite” bacteria that do not contain the plasmid, because they cannot grow as isolated colonies on ampicillin plates when they are restreaked. The true transformnants that contain the plasmid form tiny blue microcolonies that are obscured by the growth of large white satellite colonies. The predominance of very large white satellite colonies suggests that the expression of the fusion to the T4 head protein sequences is causing excessive leakage of β-lactamase from the cells, destroying ampicillin on the plates and allowing the excessive growth of non-transformed bacteria. Thus, the fusion of the head protein sequences to the truncated β-gal protein seems has the same effect on the cells as the GFP fusion above, except the manifestation is very different. Rather than allowing some of the GFP fusion to leak from the cells and make it appear to fluoresce more, leakage of proteins including β-lactamase from the cells is being manifested by an excessive growth of white satellite colonies.

Further evidence that the fusion of the head protein sequences to the truncated β-gal protein is having the same effect as the fusion to the GFPmut2 protein is that the same conditions that relieved the toxicity of the GFPmut2 fusion protein also relieved the toxicity of the truncated β-gal fusion protein. When the plates containing cells expressing the fuision to the truncated β-gal were incubated at 37° C. or higher, the colonies were blue without the excessive growth of white satellite colonies. Also, as with the GFPmut2 fusion, the groEL44 mutation in the chromosome, but not the GroEL673 mutation, allows the growth of blue colonies without the excessive growth of satellites (FIG. 4 sector II-groEL44, and sector II-GroEL673). This is as expected if, as with the GFPmut2 fusion, the groEL44 mutation relieves the toxicity of the fusion, and prevents excessive leakage of β-lactamase. The toxic effect of the fusion protein and its specific effect on cellular permeability is thus due to sequences in the head protein part of the fusion rather than in the reporter protein.

Cells were also transformed with the plasmid pGL231-176-lacZΔB, which is identical to pGL231-176Δgol-lacZΔB except that the Gol-coding region has not been deleted from the head protein sequences. This plasmid does not slow cell growth, as evidenced by the fact that normal-appearing blue colonies appear after one day of incubation at 30° C. (FIG. 4, sector I-GroEL+). Also, neither groEL mutation has any effect on the colonies.

If the toxicity of the fusion proteins is due to their being taken up by GroEL and depleting the cell of GroEL needed for other functions, then it is contemplated that fusion proteins that are too long to be taken up may be less toxic than shorter fusions. To test this, E. coli W3110 lacIq cells were transformed with the plasmid pGL231-176Δgol-lacZ in which the intact lacZ gene has been fused to the same upstream head protein sequences, expressing a much longer fusion protein. When the transformation mix was plated on LB plates containing ampicillin, Xgal, and the inducer IPTG, and the plates incubated at 30° C., the transformants were still visibly abnormal, but not as abnormal as fusions to the truncated β-gal fusion. They made almost normal-appearing blue colonies after only two days of incubation, unlike bacteria expressing the shorter fusion that never made visible colonies. Furthermore, there was no excessive growth of white satellite colonies, showing that leakage of β-lactamase from these cells was much less severe. Therefore, as predicted by the model, the longer fusion is less toxic than the shorter fusion.

The longer fusions to the intact β-gal protein are useful because they allow an estimation of the amount of fusion protein made. The head protein fusions to β-gal were made in large enough amounts to make them clearly visible. They were synthesized in only moderate amounts compared to other proteins. Therefore, the fusion proteins to the head sequences are toxic even when synthesized at only moderate levels. This is consistent with their toxicity being due to binding to GroEL, which would be a specific effect and require only relatively modest amounts of the fusion protein, rather than to a non-specific effect such as, for example, overwhelming the translational apparatus, which would presumably require much more.

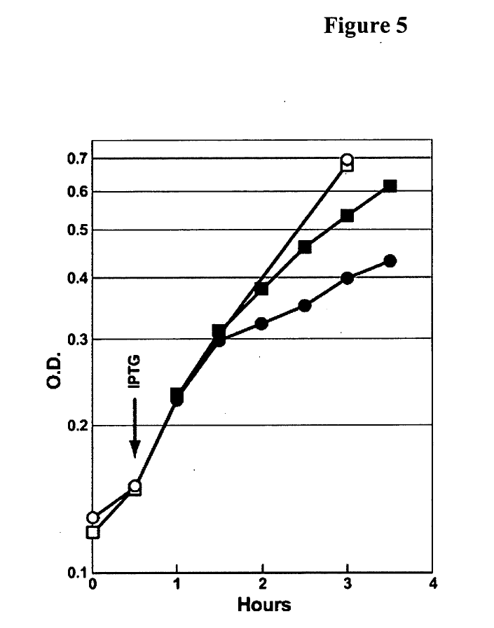

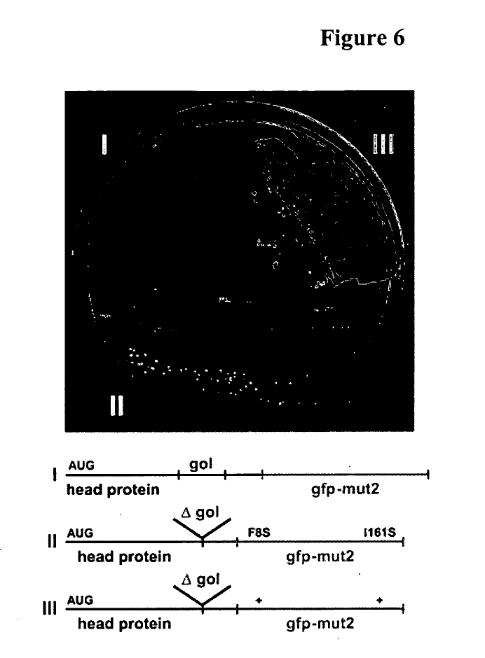

Some mutations in the GFP reporter gene may cause the fusion protein to the head protein sequences to “jam” GroEL. Another way the reporter gene product in a fusion might affect the toxicity of the fusion is if it “jams” GroEL after it is taken up. Such jamming might occur if the fusion protein is taken up but then some property of the reporter gene product in the fusion causes it not to be released as efficiently. The slower the fusion protein is released, the more severe the depletion of GroEL should be. Such jamming may explain the observation that some mutations in the GFP-coding region of a fusion can increase the toxicity of the fusion. In the process of amplifying the gfpmut2 gene for cloning, two mutations were introduced that cause the changes F8S and I161S in the GFP protein. This double mutant form of GFPmut2 does not fold properly or fluoresce under any conditions. Head protein fusions to this mutated form of GFPmut2 are more toxic to the cell than the same T4 head sequences fused to the original form of GFPmut2, as evidenced by the slower growth, after induction, of cells expressing the mutant fusion (FIG. 5). The increased toxicity of the mutant fusion protein was also apparent from the growth of transformants on plates containing the inducer IPTG (FIG. 6). Unlike cells expressing fusions to the original form of GFPmut2, which formed small, brightly fluorescing, colonies after only two days of incubation at 30° C. (FIG. 6, sector III), most of the transformants expressing the fusion of the head protein sequences to the mutant form of GFPmut2, GFPmut2F8S,I161S did not form colonies even after long periods of incubation (FIG. 6, sector II). A few brightly fluorescing colonies did eventually appear, but when the gfpmut2 gene was sequenced in the plasmid from the bacteria in one of these colonies, both mutational changes had reverted to the original sequence. This indicates that both amino acid changes in GFPmut2 contribute to the increased toxicity of the fusion. The relatively high frequency with which both mutations revert can be explained by reversion of one mutation allowing some limited growth, which is then enhanced by reversion of the second mutation.

It is further contemplated that the F8S and I161S mutational changes in GFPmut2 make the fusion protein more toxic because they make the fusion protein more likely to jam GroEL. The same mutant GFP protein is not nearly as toxic if it is synthesized autonomously or if it is fused to sequences other than the head protein sequences. The same conditions that relieve the toxicity of other fusions to the head protein sequences, such as higher temperatures or the chromosomal groEL44 mutation (see above), also relieve the toxicity of the mutant fusion protein containing GFPmut2F8S,I161 S, without significantly improving its ability to fold properly and fluoresce. This suggests that the increased toxicity of the mutant fusion protein is due to an increased ability to deplete the cell of GroEL.

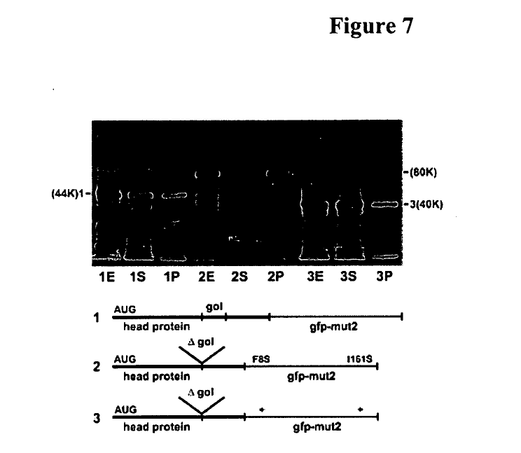

To determine how the F8S and I161S mutational changes in GFPmut2 make the fusion protein more toxic, the state of the mutant fusion protein in the cells after induction was investigated. Cells were grown at 30° C. and induced with IPTG before concentrating and lysing. The lysates were then centrifuged to pellet any precipitated proteins and Western blots with anti-GFP antibody were performed on the original lysates, the supernatants and the resuspended pellets. The results are shown in FIG. 7, lanes 2E, 2S and 2P. Most of the doubly mutant fusion protein had precipitated, and less of it had accumulated, indicating that some of it had not folded properly and been degraded. The fusion protein in the pellet did not run as a single band but as a “ladder” with most of it running larger than the unit length polypeptide. The same pattern of the fusion protein is observed if the mutant GFPmut2 protein is synthesized autonomously or as a fusion to β-gal sequences. As mentioned above, these other fusions are not toxic to the cell.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method, comprising
a) providing a vector comprising a gene encoding a heterologous protein of interest fused to a gene encoding at least a portion of a T4 head protein; and
b) introducing said protein into a cell under conditions such that said protein of interest folds into an soluble protein.

2. The method of claim 1, wherein said at least a portion of a T4 head protein comprises a gene encoding SEQ ID NO:1.

3. The method of claim 1, wherein said at least a portion of a gene encoding a T4 head protein comprises a gene encoding a portion of T4 head protein wherein said GOL region has been removed.

4. The method of claim 3, wherein said GOL region comprises amino acids 80-135 of SEQ ID NO:1.

5. The method of claim 1, wherein said cell is E. coli.

6. The method of claim 1, wherein said vector further comprises a protease cleavage site inserted in between said gene encoding a protein of interest and said gene encoding at least a portion of a T4 head protein.

7. The method of claim 1, wherein said vector further comprises a nucleic acid sequence encoding a protein purification tag, wherein said nucleic acid sequence is fused to said gene encoding a protein of interest.

8. The method of claim 7, wherein said protein purification tag is an affinity tag.

9. The method of claim 1, further comprising the step of determining the amount of soluble protein of interest.

10. The method of claim 1, further comprising the step of determining the activity of said protein of interest.

11. The method of claim 10, wherein said determining the activity of said protein of interest comprises performing an enzyme activity assay.

12. The method of claim 1, further comprising the step of performing a drug screening assay with said protein of interest, wherein said drug screening assay comprises exposing said protein of interest to a test compound and determining the activity of said protein of interest in the presence and absence of said test compound.

13. A kit comprising a vector comprising a gene encoding at least a portion of a T4 head protein fused to a site for inserting a heterologous gene of interest, wherein said gene of interest is fused to said at least a portion of a T4 head protein.

14. The kit of claim 13, wherein said at least a portion of T4 head protein comprises a gene encoding SEQ ID NO:1.

15. The kit of claim 13, wherein said at least a portion of a T4 head protein comprises a gene encoding a portion of T4 head protein wherein said GOL region has been removed.

16. The method of claim 15, wherein said GOL region comprises amino acids 80-135 of SEQ ID NO:1.

17. The method of claim 13, wherein said vector further comprises a protease cleavage site inserted in between said gene encoding a protein of interest and said gene encoding at least a portion of a T4 head protein.

18. The kit of claim 13, wherein said vector further comprises a nucleic acid sequence encoding a protein purification tag, wherein said nucleic acid sequence is fused to said gene encoding a protein of interest.

19. The kit of claim 18, wherein said protein purification tag is an affinity tag.

20. A fusion protein comprising a gene of interest fused to at least a portion of a T4 head protein.
